(19) United States
(12) Patent Application Publication
(10) Pub. No.: US 2003/0055393 A1
(43) Pub. Date: Mar. 20, 2003

(54) Consumer product lines and products comprising commonly packaged tampons which have varying absorbent capacities

(75) Inventors: Lisa Ludwig Stults, Olympia, WA (US); Jodi Elizabeth Supinski, Cincinnati, OH (US); James David Smith, Loveland, OH (US); Karen Ann Kreutz, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/238,502

(22) Filed: Sep. 10, 2002

Related U.S. Application Data
(60) Provisional application No. 60/318,341, filed on Sep. 10, 2001.

(51) Int. Cl. A61F 13/20

(52) U.S. Cl. 604/385.17; 604/904

(57) ABSTRACT

Products comprise a plurality tampons of varied absorbent capacity, tailored to meet the needs of different segments of women based on their menstrual flow. Consumer product lines comprise different types of multi-packs tailored to meet the menstrual flow needs of different segments of women based on their menstrual flow. Modularized multi-packs are presented as a way to efficiently produce different multi-packs.

### CROSS REFERENCE TO RELATED APPLICATIONS

This application claims the benefit of U.S. Provisional Application No. 60/318,341, filed Sep. 10, 2001.

### FIELD OF INVENTION

The present invention relates to products comprising a plurality of tampons which have varying absorbent capacities and are commonly packaged. The present invention also relates to consumer product lines comprising at least three products tailored to meet the menstrual flow needs of women having low, moderate, or high menstrual flow. One embodiment of the present invention utilizes modularized multi-packs as a way to efficiently produce different multi-packs.

### BACKGROUND OF THE INVENTION

A woman's menstrual cycle is typically characterized by initial light flow which subsequently increases and finally tapers off to the end of the period. Additionally, women all have different levels of overall menstrual flow throughout their cycle. In attempt to meet these varying needs, numerous absorbencies of tampons are provided in the market place. However, many women are forced to purchase more than one product to meet their varying needs throughout their cycle, or settle for inferior protection and/or some discomfort. In attempt to address this issue, Tampax® markets multi-packs configured with 10% “Juniors”, 45% “Regular” and 45% “Super” tampons sold in a common package. However, it has been discovered that while this particular configuration of multi-pack closely matches the needs of a segment of women having moderate flow relative to other menstruating women, there are other segments of women whose needs are not met by this nor by other currently available multi-pack products. These other segments include those women having lower flow or even very low flow such as those using a contraceptive pill, and those having higher flow relative to other menstruating women.

It has been discovered that while the lower flow segment benefits from a relatively high percentage of lower absorbency tampons, some may also surprisingly benefit from one or more higher absorbency tampons on their peak flow days. Alternatively, women having very low flow may not need any of the higher absorbency tampons at all. It has also been discovered that while the “high flow” segment of women need a significant number of higher absorbing tampons throughout their cycle, they also surprisingly would benefit from lower absorbency tampons for comfort on their light flow days. The use of higher absorbing tampons on light flow days is not recommended for numerous reasons, including the fact that the women may feel discomfort or even pain when removing a “dry” tampon as the tampon adheres to the vaginal tissue. Thus, none of the currently marketed multi-pack products provide an adequate selection of tampon absorbencies to meet the needs of these forgotten segments of women in a single product.

It is therefore desirable to provide a customized product, designed relative to the biophysics of the segments of women having low flow and those having high flow. Ideally, products can be tailored and provided for all low flow, moderate flow, and high flow segments of women as part of a consumer line which better meets the needs of all women. Finally, it is desirable to produce different types of multi-packs efficiently.

### SUMMARY OF THE INVENTION

This invention relates to a product comprising commonly packaged tampons wherein 100% of the tampons comprise a plurality of first tampons having a syngyna absorbent capacity of less than or equal to about 6 grams and a plurality of second tampons having a syngyna absorbent capacity of from about 6 grams to about 9 grams. This invention further relates to an additional product comprising commonly packaged tampons having at least 30% of a first tampon having a syngyna absorbent capacity of less than or equal to about 6 grams and no more than about 20% of a plurality of second tampons having a syngyna absorbent capacity of from about 9 to about 12 grams. Alternatively, this invention relates to a product comprising commonly packaged tampons having a plurality of a first tampon having a syngyna absorbent capacity of less than or equal to about 6 grams and a plurality of a second tampon having a syngyna absorbent capacity of from about 12 grams to about 15 grams. Yet another embodiment of this invention relates to a consumer product line comprising at least three multi-packs, each multi-pack comprising a plurality of tampons of varying absorbent capacity packaged in a common package and each multi-pack being tailored to meet the menstrual flow needs of women having low, moderate, or high menstrual flow. Another aspect of the present invention relates to modularized multi-packs.

### DETAILED DESCRIPTION OF THE INVENTION

As used herein the term “tampon” refers to any type of absorbent structure which is inserted into the vaginal canal for the absorption of fluid there from. Typically, tampons are constructed from an absorbent material which has been compressed in any or all of the width direction, the radial direction, and the axial direction, in order to provide a tampon which is of a size and stability to allow insertion within the vagina. See for example, U.S. Pat. No. 5,087,239 issued to Beastall et al. and U.S. Pat. No. 5,279,541 issued to Frayman et al. which are incorporated herein by reference in their entirety. As used herein the phrases “syngyna test” and “syngyna absorbent capacity” refer to the standard United States Food and Drug Administration proscribed test defining varying absorbency ranges of tampons. The use the following terminology: “Junior” (≦6 grams), “Regular” (6-9 grams), “Super” (9-12 grams), “Super Plus” (12-15 grams), and “Ultra” (15-18 grams), refers to tampons having the prescribed absorbent capacities (shown in parenthesis) provided by the U.S. Food and Drug Administration, and should not be interpreted to refer to products outside the U.S. utilizing similar terminology, but not meeting the required syngyna test requirements, unless explicitly stated. The use of such terminology herein is not intended to limit the invention to such labeling, but rather provides a convenient short hand for characterizing the desired absorbency ranges for the tampons. Providing a tampon which properly falls within these absorbency ranges requires that the total amount and type of absorbent material be controlled. Such information is readily apparent to one of ordinary skill in the art, particularly in light of the many currently marketed tampons falling within the abovementioned ranges of absorbency.

As used herein, the term “multi-pack” generically refers to products comprising two or more different absorbencies of tampons provided in a common package. As used herein, the phrase “common package” refers to a sealed package used to distribute the final tampon product. The phrase “common package” includes a unitary package housing all of the tampons of the final product being distributed/sold to the consumer; a unitary package housing smaller, individual packages or units which each house or group portions of the tampons of the final product being distributed/sold to the consumer; and/or a set of individual unitary packages grouped together to form a the final product being distributed/sold to the consumer. As such, the phrase “common package” includes but is not limited to paperboard or plastic containers or cans (with or without individual sleeves or other means for separating various absorbencies of tampons therein); and individually sealed packages which are connected together (e.g. by glue, string, shrink wrap, etc.) to form the final product being distributed/sold to the consumer. As used herein, the term “comprising” means that the various components, ingredients, or steps, can be conjointly employed in practicing the present invention. Accordingly, the term “comprising” encompasses the more restrictive terms “consisting essentially of” and “consisting of.” The percentage of tampons provided for various products is based on the total number of tampons in the common package. Other terms may be defined as they are discussed in greater detail below.

A. Commonly Packaged Tampon Products

It has been discovered that a segment of women experiencing low menstrual flow and a segment experiencing high menstrual flow cannot currently meet their menstrual flow needs throughout their entire cycle through the purchase of a single product at this time. Thus, extensive consumer research was undertaken to identify the appropriate absorbency of tampons and the appropriate number of each absorbency needed throughout these women's menstrual cycles. As a result, one aspect of the present invention provides commonly packaged products comprising tampons of varying absorbencies selected according to the needs of these segments of women.

Low Menstrual Flow Products

Multi-pack products specifically tailored to meet the menstrual flow needs of women having low menstrual flow may comprise a plurality of tampons having a syngyna absorbent capacity of less than or equal to about 6 grams (“Junior” absorbency) and a plurality of second tampons having a syngyna absorbent capacity of from about 6 grams to about 9 grams (“Regular” absorbency). In another embodiment, the multi-pack comprises at least about 30%, alternatively at least about 45% or even at least about 60%, but alternatively not more than about 95% of Junior absorbency tampons. The Regular absorbency tampons may comprise, for example, from about 5% to about 65%, alternatively, from about 15% to about 40% and in still another embodiment from about 25% to about 50% of the total tampons.

A second type of low flow product, tailored for instance for women having at least one slightly heavier flow day, may comprise a first plurality of Junior tampon in the percentage ranges shown above in addition to no more than about 20% of a plurality of second tampons which have a syngyna absorbent capacity of from about 9 to about 12 grams (“Super” absorbency). In another embodiment, these Super absorbency tampons comprise no more than about 15%, alternatively no more than about 10%, or even no more than about 5% of the total tampons. Additionally, low flow multi-packs may optionally further comprise Regular tampons in ranges as discussed above, tampons which have a syngyna absorbent capacity of from about 12 grams to about 15 grams (“Super Plus” absorbency), and/or tampons which have a syngyna absorbent capacity of from about 15 grams to about 18 grams (“Ultra” absorbency).

High Menstrual Flow Products

Multi-pack products tailored to meet the menstrual flow needs of women having high menstrual flow may comprise a plurality of tampons which have a syngyna absorbent capacity of from about 12 grams to about 15 grams (“Super Plus” absorbency). The high flow product may further comprise Junior tampons, for example comprising at least about 5%, alternatively at least about 10% or even at least about 20% or up to about 30% of the total tampons. In other embodiments, the high flow product comprises at least about 25%; alternatively at least about 35%, 50%, or 60%; or up to about 75% of Super Plus absorbency tampons.

While a high flow multi-pack preferably contains both Junior and Super Plus tampons, acceptable high flow multi-packs may not contain both Juniors and Super Plus tampons. Additionally, high flow multi-packs may also comprise a plurality of Regular, Super, or Ultra tampons. In one embodiment, the high flow multi-pack comprises at least about 10%, alternatively at least about 30% of Regular absorbency tampons. In another embodiment, the high flow multi-packs comprise from about 1% to about 65%, alternatively from about 15% to about 50%, and in yet another alternative, from about 25% to about 40% of Super and/or Ultra absorbency tampons.

In another variation of the same invention, the low and/or high flow tampon multi-pack products may be provided with other articles of manufacture such as those selected from the group consisting of learner's kits, interlabial devices, pads, panti-liners, wipes, single use heating pads, premenstrual syndrome drugs or supplements, analgesics, and mixtures thereof. Learner's kits are explained in greater detail in co-pending application Ser. No. 09/653,012, filed Sep. 1, 2000 to K. Kreutz et al.

C. Consumer Product Lines

One aspect of the present invention relates to a consumer product line comprising at least three multi-pack products tailored to meet varying menstrual flow needs of women across the span of their menstrual cycle. The consumer product lines of the present invention comprise a first multi-pack tailored to meet the menstrual flow needs of women having low menstrual flow; a second multi-pack tailored to meet the menstrual flow needs of women having moderate menstrual flow, and a third multi-pack tailored to meet the menstrual flow needs of women having high menstrual flow.

The phrase “line of consumer products” or “consumer product line” refers, in general, to a series of tampon products having a common marketing and/or technical approach. For instance, the tampon products could all be digital tampons or could all utilize the same type of applicator (e.g. plastic or paperboard, petals or open ended). Consumer product lines typically display their products in close proximity to one another on the store shelves and often use similar packaging and or nomenclature to designate a particular line. Tailoring the multi-packs to meet menstrual flow needs of various segments of women provides a greater number of women with a single product option to provide the appropriate mix of tampon products throughout their entire cycle. The term “tailored” means to customize or specially design with a set of facts in mind. The phrase “menstrual flow needs” as used herein refers both to the need to protect against menstruation leakage (e.g. staining of clothing) as well as the need for a comfortable protection device (e.g. comfort while wearing and removing). For ease of describing this concept, one segment of women are described as having “low menstrual flow”, another having “moderate menstrual flow”, and yet another as having “high menstrual flow”. The meanings of these labeled segments can be based on the women's own conception of their menstrual flow and are not intended to limit the scope of the invention particularly where a manufacturer chooses to define segments of women in different descriptive terms, but still tailor three or more multi-pack products to meet the menstrual flow needs of these segments.

Examples of multi-packs tailored to meet the needs of women having low menstrual flow are described above in the section entitled “Low Flow Menstrual Products”. Examples of multi-packs tailored to meet the needs of women having high menstrual flow are described above in the section entitled “High Flow Menstrual Products”. Examples of multi-packs tailored to meet the needs of women having moderate menstrual flow include multi-packs comprising Regular absorbency tampons combined with Junior and/or Super absorbency tampons. Optionally, Super Plus and/or Ultra tampons may also be included. A more particular example of a moderate flow multi-pack comprises from about 5% to about 25% Junior tampons, from about 25% to about 75% Regular tampons, and from about 20% to about 55% Super tampons.

Additional multi-packs may be added to the basis three product consumer line such as a “very high flow” multi-pack comprising, for example, a higher amount of Super Plus and/or Ultra tampons than the high flow multi-pack in the same line. Similarly, a the consumer line could include an additional “very low flow” multi-pack comprising only Junior and Regular tampons while the “low flow” multi-pack comprises Junior, Regular, and Super tampons.

D. Modularized Multi-Pack Products

Providing tailored multi-packs adds complexity to the production, particularly the packaging, of the tailored product given the introduction of more variables and absorbency mixes. However, in yet another aspect of the present invention, modularized multi-packs are provided which enable efficient production of different types of multi-packs. “Modularized multi-packs” are consumer product lines of multi-packs which leverage the use of modules of various absorbencies of tampons across the product line. More specifically, in the most basic form, a set of “modularized multi-packs” comprise a first multi-pack comprising at least one of a first module of first absorbency tampons and at least one of a second module of second absorbency tampons; and a second multi-pack comprising at least one of the first module of first absorbency tampons and at least one of the second module of second absorbency tampons. The first multi-pack and the second multi-pack may differ in total tampon count and tampon absorbency percentage make-up, regardless of tampon count (e.g. a first multi-pack having 35% Juniors and 65% Regulars, a second type of multi-pack having 50% Juniors and 50% Regulars). Alternatively, the first and second multi-pack differ only in absorbency count, but not percentage make-up (e.g. all multi-packs have 10% Juniors, 45% Regulars, and 45% Supers; the first multi-pack has 40 tampons: 4 Juniors, 18 Regulars, and 18 Supers; the second multi-pack has 80 tampons: 8 Juniors, 36 Regulars, and 36 Supers); or differ only in percent make-up. While the tailored products of the present invention need not be in the form of modularized multi-packs, doing so provides a way to maintain the flexibility needed to produce the specially tailored products of the present invention in a highly mechanical, mass production setting by enabling an efficient integration of multi-pack packaging cartoners with tampon converter productions lines.

In one embodiment of creating modularized multi-packs, at least two different types of multi-packs are made by forming a first module of tampons by grouping a predetermined number of a first absorbency of tampons; forming second module of tampons by grouping a predetermined number of a second absorbency of tampons; combining at least one of the first module of tampons with at least one of the second module of tampons in a common package to form a first multi-pack; and combining at least one of the first module with at least one of the second module of tampons to form a second multi-pack. “Grouping” refers to any means for accomplishing unitization of the predetermined number of a single absorbency of tampons forming the module of tampons. Grouping can be accomplished during the packaging process and the grouping means removed prior to or upon insertion of the module of tampons into the final common package forming the multi-pack. Alternatively, grouping can be accomplished by a means which is included in or as part of the final common package. For example, a receptacle which is part of the packaging equipment rather than part of the final packaged product may temporarily group a predetermined number of tampons to form a module of tampons on an assembly line. Alternatively, modules of tampons can be formed by grouping tampons in receptacles including but not limited to open sleeve cartons, tubes, canisters, plastic bags, sealed boxes or other containers, etc. Grouping may also be accomplished by binding the single absorbency tampons together with something such as film, shrink wrap, string, twine, a rubber band, releasable glue, etc.

By using this modular approach to packaging different types of multi-packs, fewer modules (and grouping means) can be used to form a wide variety of multi-packs instead of requiring completely different modules (and grouping means) for each type of multi-pack. Thus, the production plant will minimize equipment size changeovers and lines resulting in higher production reliability. This all translates into lower operating costs. More specific examples of modularized multi-packs will illustrate this efficiency, but are not intended to limit the scope of the invention.

In one example, two different types of 40 count multi-packs are formed using only two modules of tampons. The first multi-pack (e.g. a low flow multi-pack) comprises 16 Juniors (40%) and 24 Regulars (60%). The second multi-pack (e.g. a moderate flow multi-pack) comprises 8 Juniors (20%), and 32 Regulars (80%). First modules of Junior tampons are formed by grouping 8 Junior tampons together (e.g. in paperboard sleeves). Similarly, grouping 8 Regular tampons together forms a second module of tampons. The first multi-pack can then be made by combining 2 of the Junior modules of tampons with 3 of the Regular modules of tampons. The second multi-pack can be formed by combining 1 of the Junior modules with 4 of the Regular modules. Thus, the two multi-packs leverage the same modules of tampons to form completely different final products and can do so easily in a high speed setting as is readily apparent to one of ordinary skill in the art. Absent this unique method of first creating common modules of tampons for use in two different types of multi-packs, four different kinds of sleeves would have been necessary to produce these two multi-pack products; a sleeve for 16 Juniors, a sleeve for 8 Juniors, a sleeve for 24 Regulars, and a sleeve for 32 Regulars.

In yet another embodiment utilizing the modularized multi-pack approach, at least three different multi-packs are produced. A first multi-pack comprises at least one of a first module of tampons, a second multi-pack comprises at least one of the first module of tampons and at least one of a second module of tampons, and a third multi-pack comprises at least one of the second module of tampons. In the following example, the tampon modules comprise either 8 or 12 tampons. The chart below shows how many of each module are used to create the final multi-packs (e.g. “2×8” means 2 modules of 8 tampons).

| | “Low Flow” | “Moderate Flow” | “High Flow” |
|---|---|---|---|
| Junior | 12 (1 × 12) (33%) | 8 (1 × 8) (22%) | |
| Regular | 24 (2 × 12) (66%) | 12 (1 × 12) (33%) | 12 (1 × 12) (33%) |
| Super | | 16 (2 × 8) (44%) | 16 (2 × 8) (44%) |
| Super Plus | | | 8 (1 × 8) (22%) |

The following Examples illustrate the practice of the invention, but are not intended to be limiting thereof.

### EXAMPLE I

A product comprising a total of 40 tampons comprises 10 “Junior” absorbency tampons and 30 “Super Plus” absorbency tampons which are all provided in a rectangular paperboard box having a thin paperboard strip compartmentalizing the “Junior” absorbency tampons on one side and the “Super Plus” absorbency tampons on the other. Alternatively, the product is a 60 count and additionally comprises 10 “Regular” absorbency tampons and 10 “Super” absorbency tampons.

### EXAMPLE II

A product comprising a total of 40 tampons comprises 35 “Junior” (88%) absorbency tampons and 5 (13%) “Super” absorbency tampons which are provided in two separate rectangular boxes according to their absorbency and bound together in a common package with shrink wrap.

### EXAMPLE III

A product comprising a total of 30 tampons comprises 5 Regular and 15 Ultra absorbency tampons. Alternatively, the product comprises an additional 10 Super and 10 Super Plus absorbency tampons for a 50 count multi-pack. The tampons are provided in separate rectangular boxes according to their absorbency and bound together in a common package with shrink wrap.

### EXAMPLE IV

A consumer product line comprising the following three multi-pack products tailored to meet various segments of menstrual flow:

| | “Low Flow” | “Moderate Flow” | “High Flow” |
|---|---|---|---|
| Juniors | 30 | 4 | 5 |
| Regular | 10 | 18 | 5 |
| Super | | 18 | 10 |
| Super Plus | | | 20 |

Alternatively, the consumer product line additionally comprises a very high flow multi-pack comprising 5 Regular, 10 Super, and 10 Super Plus, and 10 Ultra absorbency tampons.

### EXAMPLE V

A consumer product line comprising the following three multi-pack products tailored to meet various segments of menstrual flow and optionally comprising additional articles:

| | “Low Flow” | “Moderate Flow” | “High Flow” |
|---|---|---|---|
| Juniors | 20 | 8 | |
| Regular | 20 | 20 | 10 |
| Super | | 12 | 15 |
| Super Plus | | | 15 |
| Optional | PMS supple- | PMS supple- | PMS supplements, |
| articles | ment and 2 self | ment and 4 self | analgesics, and 6 self |
| | heating, single | heating, single | heating, single use |
| | use heating pads | use heating pads | heating pads |
| | (Thermacare ®) | (Thermacare ®) | (Thermacare ®) |

Alternatively, the consumer product line additionally comprises a very low flow multi-pack comprising 30 Junior and 10 Regular absorbency tampons.

### EXAMPLE VI

A consumer product line comprising two or more of the following multi-pack products which may optionally be made with 8 count tampon modules for each absorbency:

| | | | “Moderate | |
|---|---|---|---|---|
| | “Low Flow A” | “Low Flow B” | Flow” | “High Flow” |
| Junior | 16 (2 × 8) | 16 (2 × 8) | 8 (1 × 8) | |
| | (40%) | | | |
| Regular | 16 (2 × 8) | 24 (3 × 8) | 16 (2 × 8) | 8 (1 × 8) (20%) |
| | (40%) | | | |
| Super | 8 (1 × 8) | | 16 (2 × 8) | 16 (2 × 8) (40%) |
| | (20%) | | | |
| Super | | | | 16 (2 × 8) (40%) |
| Plus | | | | |

In one embodiment of this example, all Junior tampons utilize plastic applicators and all other absorbency tampons in the multi-packs utilize paperboard applicators.

### EXAMPLE VII

A consumer product line comprising the following multi-pack products:

| | “Low Flow” | “Moderate Flow” | “High Flow” |
|---|---|---|---|
| Junior | 12 | 8 | 8 |
| Regular | 16 | 16 | 8 |
| Super | 8 | 12 | 12 |
| Super Plus | | | 8 |

### EXAMPLE VIII

A consumer product line comprising two or more of the following multi-pack products which may optionally be made with 8 and 12 count tampon modules and optionally comprising the “optional articles”:

| | “Low Flow” | “Moderate Flow” | “High Flow” |
|---|---|---|---|
| Junior | 16 (2 × 8) | 8 (1 × 8) | |
| Regular | 24 (3 × 8) | 20 (1 × 12, 1 × 8) | 12 (1 × 12) |
| Super | | 12 (1 × 12) | 16 (2 × 8) |
| Super Plus | | | 12 (1 × 12) |
| Optional | Learner's Kit and | 6 pantiliners | 2 pantiliners and |
| Articles | 6 pantiliners | | 4 Pads with wings |

### EXAMPLE IX

A production plant mass produces “moderate flow” multi-packs and “high flow” multi-packs according to Example VII. Regular tampons enter the packaging line from a Regular converter line and are placed into paperboard sleeves in two rows of four tampons to form Regular tampon modules of 8 tampons per module. Super tampons enter the packaging line from a Super converter line and are placed into paperboard sleeves of three rows of four tampons each to form Super tampon modules of 12 tampons per module. Two Regular tampon modules are combined with one Super tampon module (and 8 Junior tampons which may also be in module form) in a paper board carton common package to create the moderate flow multi-pack. One Regular tampon module is combined with one Super tampon module (and 8 Junior tampons which may also be in module form and 8 Super Plus tampons) in a paper board carton common package to create the high flow multi-pack. Optionally, low flow multi-packs may also be produced utilizing two of the Regular tampon modules and one of the Super tampon modules (and 12 Junior tampons). Because the multi-packs are modularized, the tampon converter lines are more efficiently and equally utilized across absorbencies.

### EXAMPLE X

A consumer product line comprising three different modularized multi-packs is produced. First modules of 8 Junior absorbency tampons each, second modules of 8 Regular absorbency tampons each, and third modules of 6 Super absorbency tampons each are formed on line in individual plastic wrappers for each module. A series of first multi-packs tailored to meet the needs of women having low menstrual flow are made by joining three of the first modules, one of the second module, and one of the third module of tampons in a common package. A series of second multi-packs tailored to meet the needs of women having moderate menstrual flow are made by combining one of the first module, two of the second module, and two of the third module in a common package. A series of third multi-packs tailored to meet the needs of women having high menstrual flow are made by combining two of the second module, and three of the third module in a common package. In this manner, the production line is used efficiently to create three kinds of multi-packs from only three streams of tampon modules.

### EXAMPLE XI

A consumer product line comprising two different modularized multi-packs is produced. First modules of 8 Regular absorbency tampons each and second modules of 8 Ultra absorbency tampons each are formed on line and each module bound together with a thin paper strip glued at the ends. A first multi-pack is made by placing one of the first module and four of the second module together in a paperboard container with a lid. A second multi-pack is made by placing two of the first module and eight of the second module together in a paperboard container with a lid. The first multi-pack thus contains 8 Regular (20%) and 32 Ultra (80%) tampons. The second multi-pack contains 16 Regular (20%) and 64 (80%) Ultra tampons.

What is claimed is:

1. A product comprising tampons packaged in a common package comprising:
a. a plurality of first tampons which have a syngyna absorbent capacity of less than or equal to about 6 grams; and
b. a plurality of second tampons which have a syngyna absorbent capacity of from about 6 grams to about 9 grams;
wherein the first tampons and the second tampons comprise 100% of the tampons packaged in a common package.

2. The product of claim 1 further comprising articles selected from the group consisting of pads, panti-liners, interlabial products, premenstrual syndrome products, analgesics, and single use heating pads.

3. A product comprising:
a. at least 30% of a plurality of first tampons which have a syngyna absorbent capacity of less than or equal to about 6 grams; and
b. no more than about 20% of a plurality of second tampons which have a syngyna absorbent capacity of from about 9 grams to about 12 grams;
wherein the first tampons and second tampons are packaged in a common package.

4. The product of claim 3 further comprising a plurality of third tampons which have a syngyna absorbent capacity of from about 6 to about 9 grams.

5. The product of claim 3 further comprising articles selected from the group consisting of learner's kit, pads, panti-liners, interlabial products, premenstrual syndrome products, analgesics, and single use heating pads.

6. A product comprising:
a. a plurality of first tampons which have a syngyna absorbent capacity of less than or equal to about 6 grams; and
b. a plurality of second tampons which have a syngyna absorbent capacity of from about 12 to about 15 grams;
wherein the first tampons and the second tampons are packaged in a common package.

7. The product of claim 6 further comprising a plurality of third tampons which have a syngyna absorbent capacity of from about 9 to about 12 grams.

8. A product comprising:
a. a plurality of first tampons which have a syngyna absorbent capacity of from about 6 to about 9 grams; and
b. a plurality of second tampons which have a syngyna absorbent capacity of from about 15 to about 18 grams;
wherein the first tampons and the second tampons are packaged in a common package.

9. A consumer product line comprising:
a. a first multi-pack tailored to meet the menstrual flow needs of women having low menstrual flow; and
b. a second multi-pack tailored to meet the menstrual flow needs of women having moderate menstrual flow; and
c. a third multi-pack tailored to meet the menstrual flow needs of women having high menstrual flow;
wherein the first multi-pack, the second multi-pack, and the third multi-pack each comprise a plurality of tampons of varying absorbent capacity packaged in a common package.

10. The consumer product line according to claim 9 further comprising a fourth multi-pack comprising a plurality of tampons of varying absorbent capacity packaged in a common package.

11. The consumer product line according to claim 10 wherein the fourth multi-pack is tailored to meet the menstrual flow needs of women having very high menstrual flow.

12. The consumer product line according to claim 9 wherein at least about 40% of the plurality of tampons of the first multi-pack have a syngyna absorbent capacity of less than or equal to about 6 grams.

13. The consumer product line according to claim 9 wherein no more than about 65% of the plurality of tampons of the second multi-pack have a syngyna absorbent capacity of from about 6 to about 9 grams and at least about 30% of the plurality of tampons of the second product have a syngyna absorbent capacity of from about 9 to about 12 grams.

14. The consumer product line according to claim 9 wherein no more than about 30% of the plurality of tampons of the second multi-pack have a syngyna absorbent capacity of less than or equal to about 6 grams.

15. The consumer product line according to claim 9 wherein the plurality of tampons have the same type of applicator.

16. The consumer product line according to claim 9 wherein the first multi-pack comprises at least one of a first module of tampons, the second multi-pack comprises at least one of the first module of tampons and at least one of a second module of tampons, and the third multi-pack comprises at least one of the second module of tampons.

17. The consumer product line according to claim 9 wherein
a. the first multi-pack comprises from about 35% to about 75% of tampons which have a syngyna absorbent capacity of from about 6 to about 9 grams;
b. the second multi-pack comprises from about 20% to about 60% of tampons which have a syngyna absorbent capacity of from about 9 to about 12 grams; and
c. the third multi-pack comprises from about 25% to about 65% of tampons which have a syngyna absorbent capacity of from about 12 to about 15 grams.

18. A consumer product line comprising:
a. a first multi-pack comprising at least one of a first module of first absorbency tampons and at least one of a second module of second absorbency tampons; and
b. a second multi-pack comprising at least one of the first module of first absorbency tampons and at least one of the second module of second absorbency tampons;
wherein the first multi-pack and the second multi-pack differ in a manner selected from the group consisting of total tampon count, tampon absorbency mix, and mixtures thereof.

19. The consumer product line according to claim 18 wherein the first multi-pack comprises one of the first module of first absorbency tampons and at least two of the second module of second absorbency tampons; and the second multi-pack comprises at least two of the first module of first absorbency tampons and one of the second module of tampons.

20. The consumer product line according to claim 18 further comprising a third multi-pack which comprises at least one of the first module of first absorbency tampons.
